(19) United States
(12) Patent Application Publication
(10) Pub. No.: US 2007/0129657 A1
(43) Pub. Date: Jun. 7, 2007

(54) ADJUSTABLE SHOULDER ORTHOTIC

(76) Inventor: Randall Fisher, Vail, CO (US)

Correspondence Address: SHERIDAN ROSS PC, 1560 BROADWAY, SUITE 1200, DENVER, CO, 80202, US

(21) Appl. No.: 11/567,687

(22) Filed: Dec. 6, 2006

Related U.S. Application Data
(60) Provisional application No. 60/748,069, filed on Dec. 6, 2005.

(51) Int. Cl. A61F 5/00

(52) U.S. Cl. 602/4

(57) ABSTRACT

The invention is a shoulder orthotic that provides therapeutic functional support to an injured upper limb and eliminates the weighted pressure of limb support from the injured side of the body. The functional and supportive contributions are achieved by a shoulder suspension strap that is attached to a torso belt and then laid over an unaffected shoulder where the end of a limb is suspended. The passive and active incorporation of the injured limb with the device provides relief of subluxation in the joints, allows the patient to control contraction and relaxation of limb muscles and may be adjusted to alleviate restricted blood flow in the injured limb, all of which facilitate recovery of the injured limb.

### CROSS REFERENCE TO RELATED APPLICATION

This application claims the benefit of priority under 35 U.S.C. § 119(e) to U.S. Provisional Patent Application Ser. No. 60/748,069 filed Dec. 6, 2005, which is incorporated herein by reference.

### FIELD OF THE INVENTION

The invention relates generally to orthotic devices and specifically to orthotic devices for supporting an arm while providing movement and preventing subluxation of the shoulder.

### BACKGROUND OF THE INVENTION

The human shoulder joint functions to locate the forearm, wrist and hand during activities of daily living. There are a number of conditions in which the complex coordination of the shoulder joint is lost as the result of illness or injury to the central nervous system, to the peripheral nervous system, as well as to the muscles and bony structures controlling and comprising the shoulder joint.

Shoulder weakness caused by muscle or nerve damage is often treated by immobilization of the arm in an elevated position. This treatment includes keeping the arm in the elevated position to prevent overstretching of the deltoid muscle. The deltoid muscle controls vertical abduction (elevation) of the arm. If the arm is not maintained in an elevated position, the deltoid may atrophy and may be stretched beyond its elastic limit to the point of permanent damage.

Existing shoulder braces provide both support and limited movement to injured joints and limbs. Support is typically provided through a rigid structure that adequately prevents movement of the arm, and limited movement motivated through adjustable structures that allow various components of the shoulder brace to be moved into different positions. Shoulder braces of the prior art, while providing support and limited movement, often comprise numerous metal and plastic parts and, accordingly, can be cumbersome and uncomfortable. In achieving the desired support and limitation of movement of a joint or limb, these prior art braces often focus a significant amount of the weight of the shoulder brace onto the deltoid muscles that move the injured shoulder and the nerves therein.

Orthotic devices for providing stability of the human arm at the shoulder joint include slings and splints. The shoulder sling provides only minor support to the arm and forearm, limits the use of the hand and provides no appreciable range of motion or reduction in pain or subluxation of the shoulder. The splint immobilizes the arm in a constant position, can be used for only short periods of time due to the pressure applied to the pelvic bone, and limits the mobility of the joint in a way that may become permanent if used for long periods.

Orthotic devices for providing mobility of the shoulder joint include mobile arm supports and functional arm braces. The mobile arm supports are attached to wheelchairs or beds and are therefore useful only for the non-ambulatory patient. Functional arm braces allow certain motions of the injured shoulder but fail to provide sufficient stability, especially when spasticity is a factor.

Thus, there is a long felt need for a shoulder orthotic that provides stable support to an upper limb for the mobile patient. Preferably, the orthotic would not support the weight of the upper limb on the injured side of the body. The ideal orthotic would allow passive and active movement of the injured limb for the relief and prevention of subluxation and simultaneously allow the patient to control contraction and relaxation of limb muscles to enhance the recovery of an injured limb.

### BRIEF DESCRIPTION OF THE DRAWINGS

FIG. 1 depicts a shoulder orthotic of the present invention.

FIG. 2 is a schematic drawing of a patient wearing the shoulder orthotic of FIG. 1.

FIGS. 3A, 3B and 3C show additional embodiments of the present invention.

FIG. 4 is a schematic drawing of a patient wearing one embodiment of the shoulder orthotic of the present invention including a static or non-flexible shoulder suspension strap.

FIG. 5 is a schematic drawing of a patient wearing one embodiment of the shoulder orthotic of the present invention including an elastic suspension member between the shoulder suspension strap and the torso belt.

FIG. 6 is a schematic drawing of the front of a patient wearing one embodiment of the shoulder orthotic of the present invention including an elastic shoulder suspension strap.

FIG. 7 is a schematic drawing of the back of a patient wearing one embodiment of the shoulder orthotic of the present invention including an elastic shoulder suspension strap.

FIG. 8 depicts a shoulder orthotic of the present invention including a variable length strut.

FIG. 9 is a schematic drawing of a patient wearing the shoulder orthotic of FIG. 8.

### SUMMARY OF THE INVENTION

The present invention is drawn to a shoulder orthotic and to methods of manufacturing and using the orthotic device. The orthotic provides therapeutic functional support to an injured upper limb while eliminating the weighted pressure of limb support from the injured side of the body. The functional and supportive contributions are achieved via an adjustable elastic component attached to a shoulder suspension strap laid over an unaffected shoulder to suspend the hand or end of limb. The passive and active incorporation of the injured limb with the orthotic provides relief of subluxation in the joints, and allows the patient to control contraction and relaxation of limb muscles, all of which may facilitate recovery of the injured limb.

The shoulder orthotic of the present invention includes a torso belt that is worn around the torso of the patient. The torso belt can be clasped around the torso of the patient at any point above the waist and below the shoulders of the patient. The torso belt can be composed of any suitable material that can be firmly affixed around a patient's torso and can support the weight of the patient's arm. Preferably, the torso belt is composed of a rigid material such as plastic, leather or metal such that the torso belt can be more easily attached to, and removed from, the patient's torso. Preferably, the torso belt is configured to be secured around the patient's torso at the waist.

The torso belt may be secured around the patient's torso by any means suitable to prevent unintended release of the torso belt. Examples of suitable closures include, but are not limited to, a buckle, hook-and-loop (for example VELCRO™) closures, button and button hole(s), zipper(s), one or more string ties, a hook and islet, a D-ring and clasp, magnets, shock cord, snaps and the like. Preferably, the torso belt includes a means for securing the belt around the patient's waste in a way that can be easily opened and closed with one hand, making the belt and the shoulder orthotic of the present invention easily secured and removed by a patient with one injured upper limb.

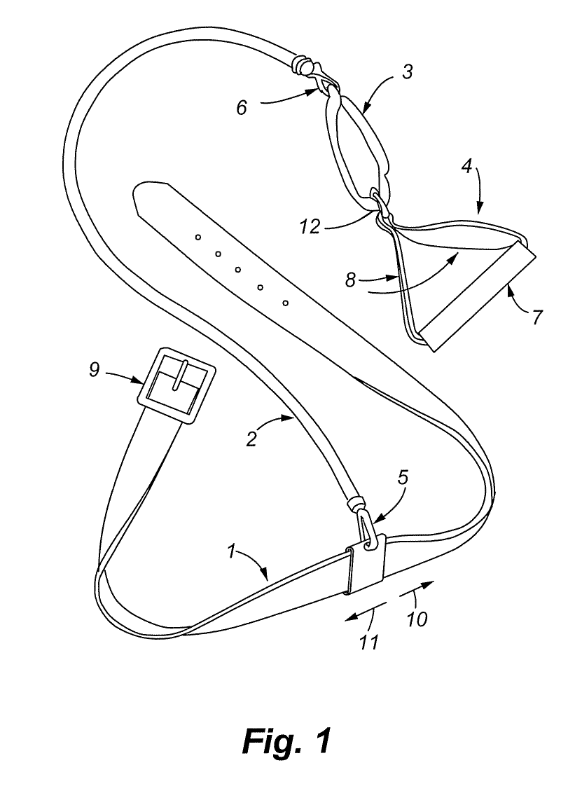

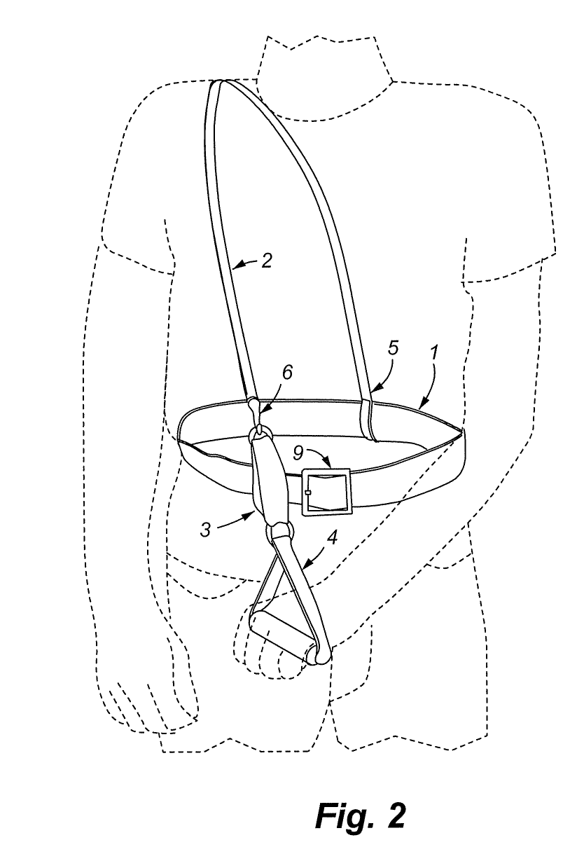

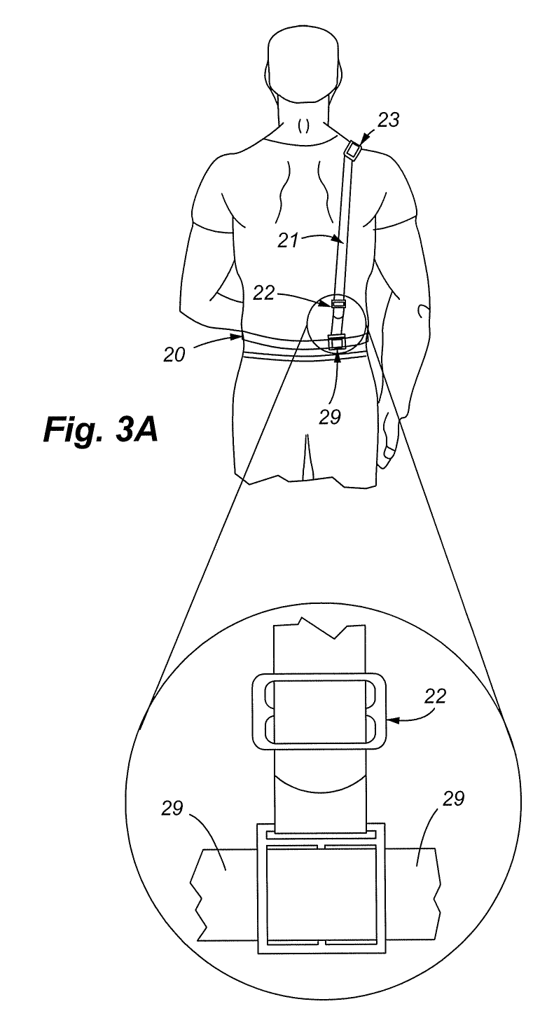

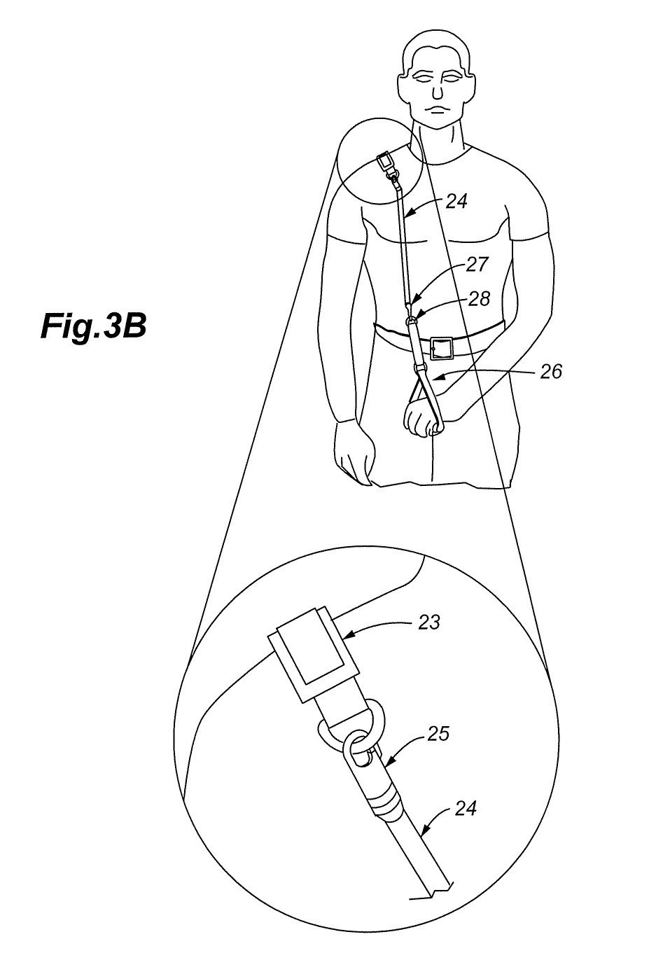

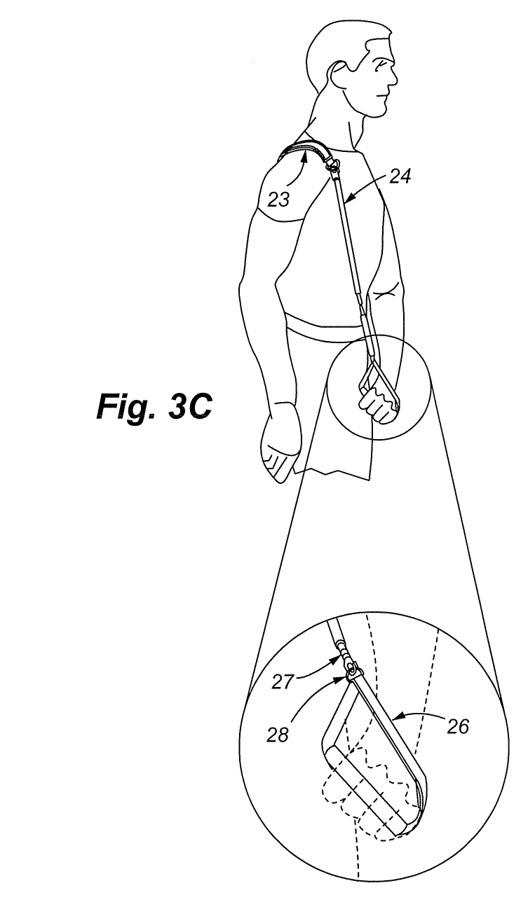

The shoulder orthotic of the present invention also includes a shoulder suspension strap attached to the torso belt. The shoulder suspension strap is configured to attach to the torso belt behind the patient wearing the shoulder orthotic and extend over an uninjured shoulder of the patient to a point below the patient's collar bone. The shoulder suspension strap preferably extends to a length that allows the patient to easily grasp an end of the shoulder suspension strap with the limb extending from an injured shoulder. Preferably, the shoulder suspension strap is adjustably-attached to the torso belt such that the shoulder suspension strap can be moved along the length of the torso belt at the point of attachment to the torso belt. In this way, the shoulder suspension strap can be moved along the length of the belt as required for comfort or to conform to positioning changes required by the patient's therapy. Additionally, this allows for the use of the device by patients recovering from injuries of either shoulder by simply adjusting the point of attachment between the shoulder suspension strap and the torso belt to allow the shoulder suspension strap to rest over an uninjured shoulder. Preferably, the shoulder suspension strap is releasably-attached to the torso belt to allow quick removal of the shoulder suspension strap from the torso belt. In one embodiment shown in FIGS. 1 and 2, the shoulder suspension strap is of a length that allows the shoulder suspension strap to extend well below the patient's collar bone to as low as the patient's waist. In another embodiment shown in FIGS. 3B and 3C, the shoulder suspension strap terminates at a point nearly level with the patient's collar bone.

The shoulder suspension strap may be composed of any suitable material to support the arm of a patient. Suitable materials include, but are not limited to, leather, rubber, cloth, nylon webbing, vinyl, urethane and the like. In one preferred embodiment shown in FIGS. 1 and 2, the shoulder suspension strap is an elastic material that can support the weight of the patient's arm while simultaneously flexing against tension exerted by the patient's arm in a direction away from the patient's un-injured shoulder, over which the shoulder suspension strap resides. In another embodiment shown in FIGS. 3A, 3B, 3C, 4 and 5 the shoulder suspension strap is made of a non-flexible material that is suitable to support an injured limb from the shoulder of the opposite (un-injured) side of the body. The length of this shoulder suspension strap may be adjusted to correctly position the end of the shoulder suspension strap that terminates in front of the patient. Any useful means of adjusting the length of the shoulder suspension strap may be used. A preferred length adjustment means is shown in FIG. 3A in which a length adjustment slide is depicted allowing the shoulder suspension strap to be adjusted in length. In this embodiment, one end of the shoulder suspension strap is attached to an elastic suspension strap that can support the weight of the patient's arm while simultaneously flexing against tension exerted by the patient's arm in a direction away from the patient's uninjured shoulder.

In these embodiments, the patient can exert pressure against the elastic strap by flexing either or both of the triceps muscle on the arm and the deltoid muscles of the injured shoulder. This voluntary movement and exercise of the arm and shoulder muscles forces the bones of the shoulder joint together and strengthens the arm and shoulder muscles. These effects function to prevent atrophy of the arm and shoulder muscles as well as subluxation of the shoulder joint due to atrophy of these muscles. Subluxation is associated with mild to profound pain on the part of the recovering patient and therefore, preventing or decreasing subluxation of this joint aids in recovery by decreasing pain and contributing to the patient's overall well being. In preferred embodiments of the present invention, the elastic strap is an elastic tube that may be releasably-attached to the torso belt or to a non-elastic shoulder suspension strap through a quick-release metal clip.

In a preferred embodiment of the present invention, the shoulder orthotic includes a handle grip attached to the end of the shoulder suspension strap opposite the end attached to the torso belt. The handle grip may take the form of any grip or attachment that can be grasped by, or connected to, the hand or wrist (that may be in a cast or splint) or the end of an upper limb of a patient. Preferably, the handle grip is a rounded handle with a straight grip that can be comfortably gripped by a patient's hand. In a preferred embodiment shown in FIG. 3B, the handle grip is provided by a triangular attachment to one end of the shoulder suspension strap. In this preferred embodiment, two sides of the triangular attachment are flexible while one side is rigid, and it is this rigid side that is intended to be gripped by the patient's hand. Suitable handle grips may be provided by molded hand grips, bicycle handle grips, rings of varying sizes through which a patient may insert several fingers or the end of an upper limb, spheres of different sizes that may be gripped by the patient's hand, hour glass-shaped materials that can be griped by the patient's hand, ergonomically-shaped handles configured to be gripped and squeezed by the patient's hand, a glove fabricated to receive the patient's hand and attached to the shoulder suspension strap through a quick-release attachment, and the like.

Optionally, the shoulder orthotic of the present invention may include an adjustable length attachment for lengthening or shortening the shoulder suspension strap. By adjusting the length of the shoulder suspension strap, this adjustable length attachment can be used to effectively position the end of the shoulder suspension strap that is gripped by the patient to hang closer to, or further from, the patient's collar bone. In the embodiment of the present invention including a handle grip, the adjustable length attachment can be used to effectively position the handle grip closer to, or further from, the patient's collar bone. The adjustable length attachment may be included in the shoulder orthotic of the present invention between the torso belt and the shoulder suspension strap. Alternatively, the adjustable length attachment may be included at the opposite end of the shoulder suspension strap, i.e. the end of the shoulder suspension strap that is gripped by, or is otherwise attached to, the patient's limb. In the embodiment of the present invention including a handle grip, the adjustable length attachment may be located between the shoulder suspension strap and the torso belt. Optionally, adjustable length attachments may be included at both ends of the shoulder suspension strap.

In the embodiment of the present invention having a shoulder suspension strap of a length that terminates near the patient's collar bone, the shoulder suspension strap is preferably made of a non-elastic material and is attached to an elastic suspension member that is connected at one end to the shoulder suspension strap and at the opposite end to a handle or to an adjustable length attachment. The length of this elastic suspension member may be adjusted to best position the patient's arm in front of the body. The elastic suspension member may be made of differing materials or of various sizes of the same material to allow for differences in tension and elasticity of the elastic suspension member. In a preferred embodiment, the elastic suspension member is elastic tubing that can be cut to a desired length to adjust the position of the patient's arm in front of the patient's body. This elastic tubing may be supplied in different tube diameters to allow for different elasticity characteristics in the elastic suspension member.

If present, an adjustable length attachment may be any attachment to the shoulder suspension strap that can be shortened or lengthened and then fixed at a length at which it is capable of supporting the weight of a patient's upper limb. Preferably, the adjustable length attachment can be quickly and easily adjusted in length while maintaining the ability to support the weight of a patient's upper limb. Exemplary devices that may be used as an adjustable length attachment include, but are not limited to, hook-and-loop closures, pull blocks, wind-able cords, shock cords, nylon webbing, buckled belts, adjustable straps, and the like. Preferably, the adjustable length attachment is nylon webbing with a hook-and-loop closure surface.

An optional shoulder pad may be used to cushion the weight placed against the un-injured shoulder by the shoulder suspension strap. The shoulder pad may be attached to the shoulder suspension strap and in a preferred embodiment; the shoulder suspension strap is threaded through attachments to the shoulder pad or through a sleeve-like shoulder pad to allow the position of the shoulder pad to be adjusted along the length of the shoulder suspension strap. The shoulder pad may be composed of any suitable cushioning material that will effectively disperse pressure from the shoulder suspension strap on the patient's un-injured shoulder.

Optionally, the shoulder orthotic of the present invention may include an adjustable arm brace strut with a hand grip. The arm brace strut is incorporated into the arm sling of the present invention by attaching to the torso belt and extending therefrom to a position in front of the patient. A hand grip is associated with the end of the strut and may be attached in a rigid or flexible or adjustable manner. The end of the strut that is opposite the end attached to the torso belt is attached to the shoulder suspension strap, which is made of an elastic material, thereby allowing the patient to move and flex the arm and shoulder muscles while grasping the hand grip to prevent subluxation as described earlier. Using this strut incorporated into the arm sling, the patient reaches forward with an arm and grasps the hand grip. The strut and associated hand grip provide support to elevate and position the arm in front of, and to the side of, the patient—thereby holding the arm away from the body at the elbow. This prevents the arm from resting against the trunk of the patient's body.

In instances in which the patient's arm is partially or completely deinervated, or not strong enough to hold the arm away from the trunk, the deinverated extremity may not function in many ways that may include normal vascular function. When a deinervated or weakened arm is cradled against the patient's trunk in a conventional arm sling, the arm may be pinched or uncontrollably pressed by gravity to the patient's trunk, causing a restriction in the blood flow to the affected shoulder/arm. As a result of the restricted blood flow, the recovery of the arm and/or shoulder may be reduced or require a substantially longer time of recovery. Thus, the embodiment of the present invention incorporating the strut and associated hand grip prevents the restricted blood flow in this patient population and aids in recovery. Similarly, other conditions that lead to vascular complications in the extremities may be aided by the use of the arm sling of this embodiment that eliminates the restricted blood flow of conventional arm slings.

The arm sling incorporating the strut and associated hand grip may also be used for the recovery of strength and coordination following injuries that limit the patient's ability to control the arm in response to external forces. For example, up and down forces of momentum that occur when jogging or walking to position an arm for the balance and counter-balance required in these physical activities may require maintaining the arm in a 90-degree “runners position” allowing front and back (extension and return) of the arm. This is the typical balance and counter-balance component of running that is normally contributed by the arms in conjunction with the legs. The arm strut attached to the elastic shoulder suspension strap can maintain arm position while allowing an adjustable degree of extension and resistance, which also enhances the recovery of strength and coordination, even in instances in which blood flow in the arm and shoulder is not restricted.

The strut and associated hand grip can be optionally added to, or removed from, the adjustable shoulder orthotics of the present invention described above as an alternate to the handle grips. The strut is attached at one end to the torso belt and at the opposite end to the end of the shoulder suspension strap. Preferably, the strut is adjustable in length and most preferably, the strut is telescopic, formed from a combination of multiple tubes made of any suitable rigid material(s), dimensioned to position an outer tube housing an series of one or more inner tubes in sequence to provide a telescopic extension. The strut's telescopic range of action may be adjustable by varying the length and number of telescopic sections which may be fixed, or preset and changeable by a or other suitable mechanical mechanism such as a stop pin, keyed compression ring, or clamp. Resistance of the telescopic action may be controlled by friction between the nested tubes, induced by any known method such as a mechanical threaded and keyed compression ring, interchangeable internal spring(s) of various resistances, or pressurized cylinder(s).

One end of the strut is fastened to the torso belt by a mechanical connective device. The opposite end of the strut includes a handle grip. Preferably, the handle grip can be released from the strut via a release mechanism. The top of the handle grip is attached to the shoulder suspension strap described above. Preferably, the handle grip includes a D-ring anchor for receiving a snap ring on the shoulder suspension strap.

As described above, the handle grip associated with the strut may take the form of any grip or attachment that can be grasped by, or connected to, the hand or wrist (that may be in a cast or splint) or the end of an upper limb of a patient. Preferably, the handle grip is a rounded handle with a straight grip that can be comfortably gripped by a patient's hand.

The strut may be attached to the belt with a single point connection that provides more freedom of motion of the strut by pivot. Alternatively, the strut may be attached to the belt using a larger point, or multiple points of connection, to the torso belt providing control over unwanted vertical, lateral or rotational effects.

### DETAILED DESCRIPTION OF PREFERRED EMBODIMENTS OF THE INVENTION

Additional objects, advantages, and novel features of this invention will become apparent to those skilled in the art upon examination of FIGS. 1-7 and the following description of the preferred embodiments shown therein, which are not intended to be limiting. FIG. 1 shows a torso belt (1) having a buckle clasp (9). The torso belt is releasably-attached to an elastic shoulder suspension strap (2) through a quick-release clip (5). The embodiment shown in FIG. 1 includes an adjustable length attachment (3) in the form of a loop of nylon webbing that can be adjusted and fastened with hook and loop closure material. The adjustable length attachment (3) is releasably-attached to the elastic shoulder suspension strap (2) with a quick-release clip (6). A handle grip (4) is attached to the adjustable length attachment (3) at a D-ring (12). The handle grip (4) is triangular in shape having two sides (8) of the triangular form made of flexible nylon webbing and the remaining side (7) being an easily-gripped rigid material.

As shown in FIG. 1, the point of attachment of the elastic shoulder suspension strap (2) to the torso belt (1) can be adjusted along the length of the torso belt (1) in a direction towards the buckle (11) or a direction away from the buckle (10).

FIG. 2 depicts the orthotic of FIG. 1 in place on a patient.

FIGS. 3A, 3B and 3C show a torso belt (20) configured to attach to the patient's waist and a shoulder suspension strap (21) that is attached at one end to the torso belt (20) and is adjustable in length through adjustment means (22) to terminate near the patient's collarbone. The shoulder suspension strap (21) is threaded through an optional shoulder pad (23). One end of the shoulder suspension strap (21) is attached to an elastic suspension member (24) through a quick-release connection (25). The opposite end of the elastic suspension member (24) is connected to a handle (26) through a quick release connection (27) attached to a D-ring (28) on the handle (26). One end of the shoulder suspension strap (21) is attached to the torso belt (20) via a belt slide (29) that allows the position of the shoulder suspension strap (21) to be adjusted along the length of the torso belt (20).

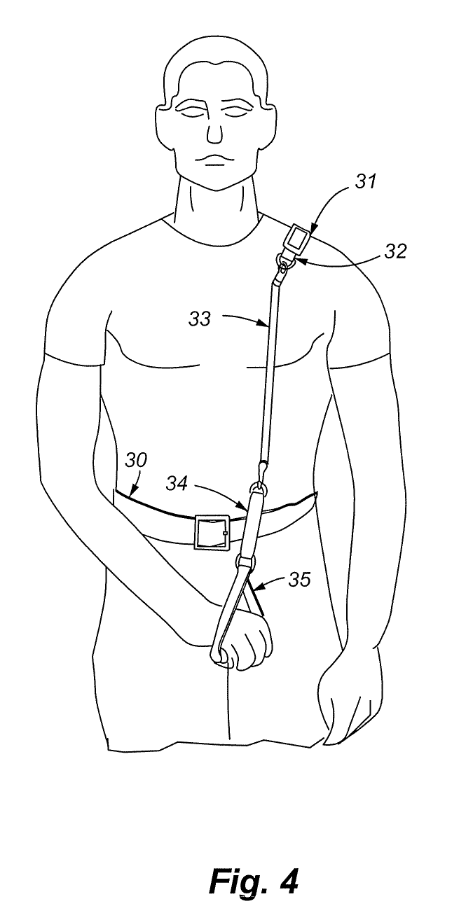

FIG. 4 shows a torso belt (30) that is connected to a shoulder suspension strap (32), which rests on a shoulder pad (31) on a patient's un-injured shoulder. The patient grips the handle (35) that is attached to an elastic suspension member (33) through an adjustable length attachment (34). In this embodiment, the shoulder suspension strap (32) is not made of an elastic material but the elastic suspension member (33) provides the flexibility that allows the patient to exercise the shoulder and arm muscles of the injured limb by exerting downward pressure against the handle (35).

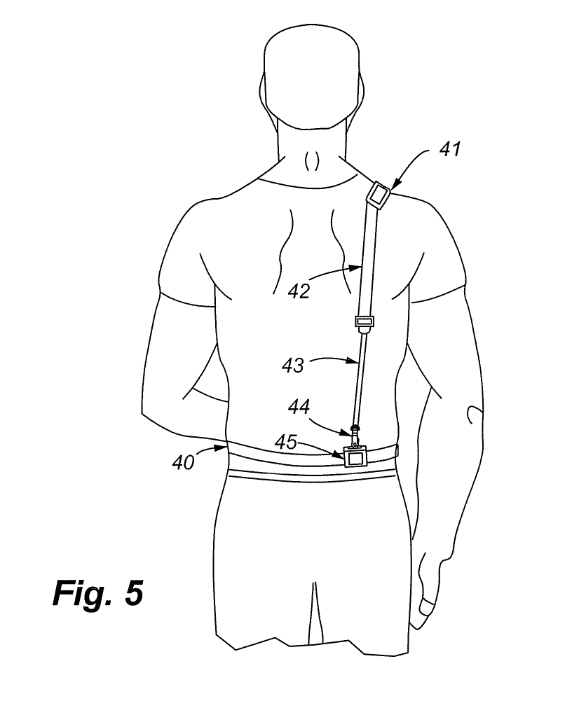

FIG. 5 shows a torso belt (40) that is connected to a shoulder suspension strap (42), which rests on a shoulder pad (41) on a patient's uninjured shoulder. The shoulder suspension strap (42) is attached to the torso belt through an elastic suspension member (43) that is connected to the torso belt (40) through a quick release clip (44) attached to an adjustable attachment (45) that can be moved along the length of the torso belt (40). In the embodiment of the present invention shown in FIG. 5, the elastic suspension member (43) residing between the shoulder suspension strap (42) and the torso belt (40) provides flexibility in the shoulder orthotic to allow the patient to bend at the waist while maintaining the correct positioning of the shoulder suspension strap (42) and shoulder pad (41) on the uninjured shoulder of the patient.

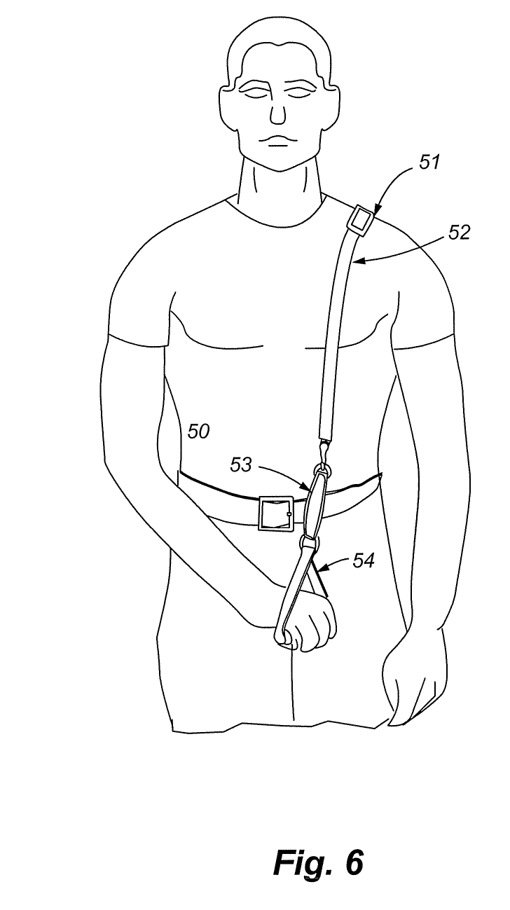

FIG. 6 shows an embodiment of the present invention in which a torso belt 50 is attached to a shoulder suspension strap (52). The shoulder suspension strap (52) resides on the patient's uninjured shoulder on a shoulder pad (51) and is attached to a handle (54) through an adjustable length attachment (53). In this embodiment, the shoulder suspension strap (52) is made of an elastic material that provides the flexibility for a patient to exercise the shoulder and arm muscles of the injured limb by exerting downward pressure against the handle (54) without requiring an additional elastic suspension member.

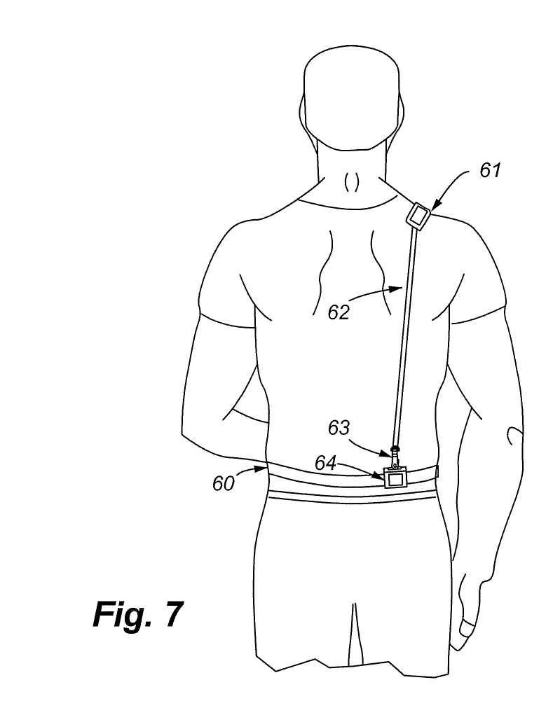

FIG. 7 shows a torso belt (60) that is connected to a shoulder suspension strap (62), which rests on a shoulder pad (61) on a patient's un-injured shoulder. The shoulder suspension strap (62) is attached to the torso belt through a quick release clip (63) attached to an adjustable attachment (64) that can be moved along the length of the torso belt (60). Using the shoulder orthosis shown in this embodiment of the present invention, the shoulder suspension strap (62) is made of an elastic material that provides flexibility to allow the patient to bend at the waist while maintaining the correct positioning of the shoulder suspension strap (62) and shoulder pad (61) on the uninjured shoulder of the patient.

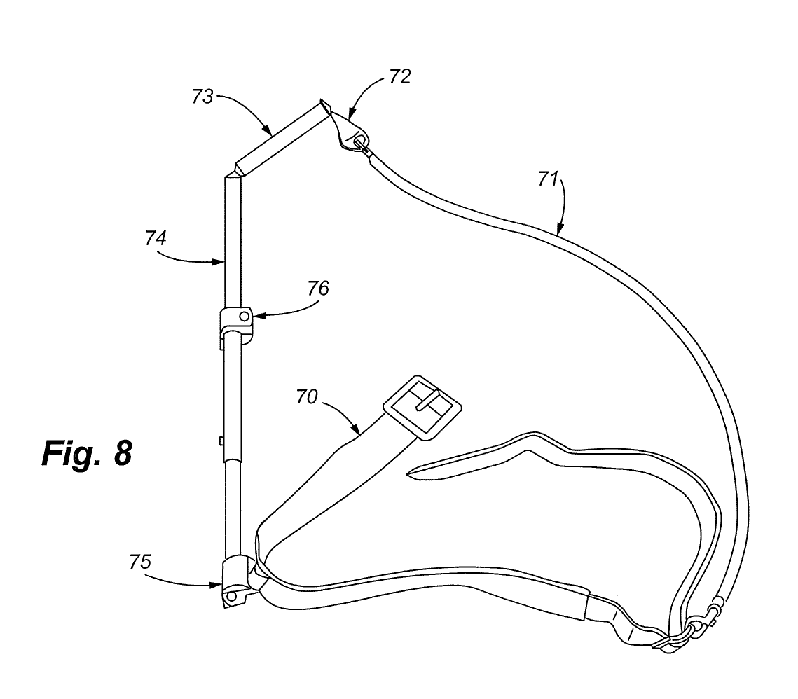

FIG. 8 shows a torso belt (70) releasably connected to an elastic shoulder suspension strap (71) that is clasped to a cylindrical handle grip (73) through a flexible D-ring attachment (72). The handle grip (73) is flexibly connected to one end of a telescopic strut (74) that is attached at an opposite end to the torso belt (70) through a single point connection (75). The position of the single point connection (75) may be adjusted along the length of the torso belt (70) to allow for control over the position and lateral movement of the strut (74). The positioning of the strut (74) may also be adjusted by lengthening or shortening the strut (74) by locking the telescoping tubes at a desired length using the slide resistor and stop (76).

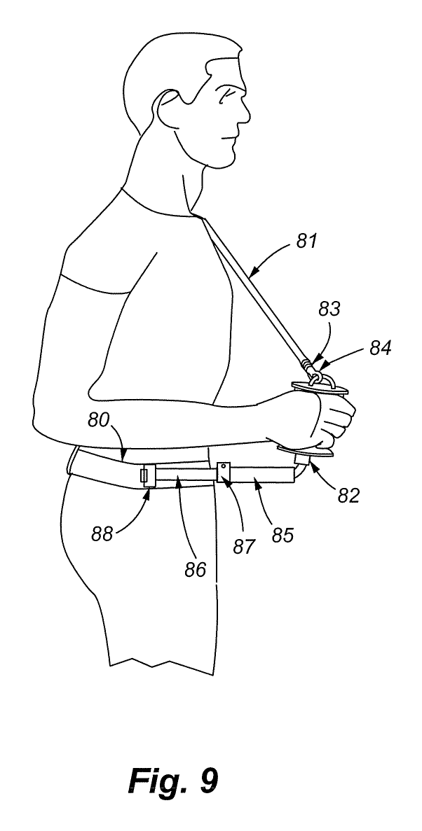

FIG. 9 shows one embodiment of the shoulder orthosis of the present invention that includes a strut, positioned on a patient. The torso belt (80) is positioned around the patient's waist and is releasably connected to an elastic shoulder suspension strap (81) positioned over the patient's shoulder, opposite the injured limb/shoulder. The shoulder suspension strap (81) is connected to a handle grip (82), which is interchangeable to allow the use of multiple handle types, through a snap hook (83) clasped to a D-ring (84) on the handle grip (82). The handle grip (82) is flexibly connected to a telescopic strut composed of an outer tube (85), an inner tube (86) and a mechanical slide resistor (87) used to lock the two tubes (85 and 86) in place at a desired length. The inner tube (86) is secured on the torso belt (80) through a single connection (88) that may be adjusted in position along the length of the torso belt (80).

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. An orthotic comprising:
a torso belt; and,
a shoulder suspension strap attached to the torso belt and having a length sufficient to extend from the torso belt, over a patient's shoulder, to a position below the patient's collar bone.

2. The orthotic of claim 1, comprising a hand grip attached to the shoulder suspension strap at an end of the shoulder suspension strap opposite the attachment to the torso belt.

3. The orthotic of claim 2, wherein the attachment of the hand grip to the shoulder suspension strap comprises an adjustable length attachment.

4. The orthotic of claim 3, wherein the adjustable length attachment of the hand grip to the shoulder suspension strap comprises a length-adjusting closure selected from the group consisting of snaps, hook-and-loop fabric closure, a zipper, a buckle, shock cord, nylon webbing and a wind-able cord.

5. The orthotic of claim 1, wherein the attachment of the shoulder suspension strap to the torso belt comprises an adjustable length attachment.

6. The orthotic of claim 1, wherein the attachment of the shoulder suspension strap to the torso belt comprises an elastic suspension member.

7. The orthotic of claim 1, wherein the torso belt comprises a closure to secure the torso belt around a patient's torso, the closure selected from the group consisting of a buckle and islet clasp, a hook-and-loop fabric closure, tie strings, and a hinge and clasp.

8. The orthotic of claim 1, wherein the shoulder suspension strap comprises an elastic tube.

9. The orthotic of claim 1, wherein the shoulder suspension strap is releasably-attached to the torso belt.

10. The orthotic of claim 1, wherein the shoulder suspension strap is releasably-attached to a handle grip.

11. The orthotic of claim 1, wherein the shoulder suspension strap is releasably-attached to a handle grip at an end of the shoulder suspension strap opposite the adjustable attachment to the shoulder suspension strap.

12. The orthotic of claim 16, wherein the handle grip is selected from the group consisting of a triangular handle having two flexible sides and one rigid side, a bicycle handle grip, a ring through which a patient may insert several fingers or the end of an upper limb, a sphere that may be gripped by the patient's hand, an hour glass-shaped material that can be griped by the patient's hand, an ergonomically-shaped handle configured to be gripped and squeezed by the patient's hand, and a glove fabricated to receive the patient's hand.

13. The orthotic of claim 1, further comprising an adjustable length attachment.

14. The orthotic of claim 13, wherein the adjustable length attachment is attached to one end of the shoulder suspension strap.

15. The orthotic of claim 13, wherein the adjustable length attachment connects one end of the shoulder suspension strap and a handle grip.

16. The orthotic of claim 1, further comprising an elastic suspension member attached at one end to a non-elastic shoulder suspension strap, and at an opposite end to a handle.

17. The orthotic of claim 1, further comprising a shoulder pad connected to the shoulder suspension strap.

18. The orthotic of claim 1, further comprising a strut extending from a connection point on the torso belt to a connection to the shoulder suspension strap, wherein the connection comprises a handle grip.

19. The orthotic of claim 18, wherein the strut is a telescopic strut comprising at least two nested tubes and a mechanical slide resistor and stop adapted to lock the telescopic strut at variable lengths.

20. An orthotic comprising:
a torso belt;
an elastic shoulder suspension strap attached at one end to the torso belt and having a length sufficient to extend from the torso belt, over a patient's shoulder, to a position below the patient's collar bone;
a hand grip attached to the shoulder suspension strap at an end of the shoulder suspension strap opposite the end attached to the torso belt;
an adjustable length attachment connecting the end of the shoulder suspension strap opposite the end attached to the torso belt and a handle grip; and,
a shoulder pad connected to the shoulder suspension strap.

21. An orthotic comprising:
a torso belt;
an elastic shoulder suspension strap attached at one end to the torso belt and having a length sufficient to extend from the torso belt, over a patient's shoulder, to a position below the patient's collar bone;
a hand grip attached to the shoulder suspension strap at an end of the shoulder suspension strap opposite the end attached to the torso belt;
an adjustable length attachment connecting the end of the shoulder suspension strap opposite the end attached to the torso belt and a handle grip;
a shoulder pad connected to the shoulder suspension strap; and,
a variable length strut extending from a connection point on the torso belt to a connection to the hand grip attached to the shoulder suspension strap.
